(19) United States
(12) Patent Application Publication
(10) Pub. No.: US 2010/0298703 A1
(43) Pub. Date: Nov. 25, 2010

(54) VARIABLE LINE LENGTH SCANNING PATTERNS

(75) Inventor: Dave Prater, Andover, MA (US)

Correspondence Address: PHILIPS INTELLECTUAL PROPERTY & STANDARDS, P.O. BOX 3001, BRIARCLIFF MANOR, NY, 10510, US

(73) Assignee: KONINKLIJKE PHILIPS ELECTRONICS N.V., EINDHOVEN (NL)

(21) Appl. No.: 12/445,768

(22) PCT Filed: Oct. 18, 2006
(86) PCT No.: PCT/IB07/54252

§ 371 Date: Apr. 16, 2009

(51) Int. Cl. A61B 8/14

(52) U.S. Cl. 600/443

(57) ABSTRACT

Methods and apparatus are provided for improved image acquisition rates in medical imaging compared to that of sector scanning methods using lines of constant length absent variable length lines. An embodiment includes scanning a central angular region of a volume of interest using a plurality of fixed line lengths and scanning outside of the central angular region of the volume of interest using a plurality of variable length lines.

The technical field of the application is methods and apparatus for high speed image acquisition in medical imaging.

Ultrasound is a noninvasive, easily portable and relatively inexpensive tool for medical imaging. These attributes have led to the wide scale adoption of ultrasound imaging of a variety of organs. In particular, ultrasound is often used to detect motion, for example, the movement of a fetus or an organ such as the heart, to detect potential pathologies. Such imaging of the heart, referred to as echocardiography, poses particular challenges because diagnosis of some heart problems, such as mitral valve prolapse, involve analyzing movements of the heart that are subtle and can be detected only if the imaging device can acquire images rapidly.

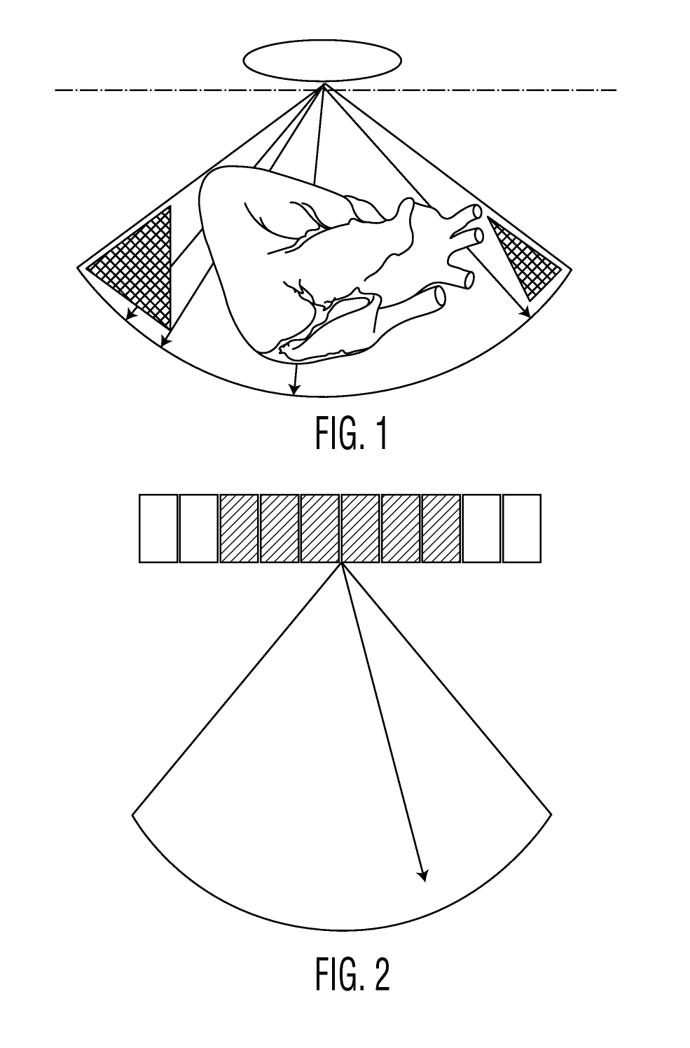

In ultrasound scanning, time resolution is dictated by how long it takes for echoes from the deepest tissues to return to the transducer, as described in Andrew R. Webb, Introduction to Biomedical Imaging 131 (2003), which is hereby incorporated herein by reference. Conventional ultrasound imaging systems scan polar sectors that have a small, but uniform (fixed) length radius, in order to obtain full imaging of the volume of interest. However, such fixed line length scanning is inefficient in many cases because regions outside of the volume of interest are also scanned, increasing the amount of time required in order to obtain each individual image and thereby lowering the image acquisition rate. This inefficient aspect of fixed line length scanning in echocardiography is illustrated in FIG. 1.

An embodiment of this invention provides a method of imaging of a medical subject including scanning a central angular region of a volume of interest using a plurality of fixed length lines and scanning outside of the central angular region of the volume of interest using a plurality of variable length lines, thereby acquiring an image at an improved acquisition rate compared to that of sector scanning methods using lines of constant length absent variable length lines.

In a further embodiment, scanning the central angular region and/or scanning outside the central angular region further includes a non-sequential pattern. In a further embodiment, the non-sequential pattern includes alternating an angular position of a beam or a pulse from a first side of the volume of interest to a second side of the volume of interest.

In another embodiment, which is exemplary and not to be further limiting, scanning the central angular region includes scanning with constant length lines between the angles of about −15 degrees to about +15 degrees, for example, at least about ±10 degrees, ±20 degrees or ±25 degrees. In another embodiment, also exemplary and not to be further limiting, scanning outside the central angular region includes scanning with variable length lines between angles of about −45 degrees to about −15 degrees and about +15 to about +45 degrees, for example, at least about ±10 degrees, or ±20 degrees, or ±25 degrees, or ±30 degrees, or ±35 degrees, or ±40 degrees, or ±50 degrees, or ±55 degrees.

In another embodiment, which is exemplary and not to be further limiting, the fixed length lines have a length between about 3 cm and about 30 cm. In another embodiment, also exemplary and not to be further limiting, the fixed length lines have a length of about 16 cm for example, at least about, 4, or 6, or 8, or 10, or 12, or 14, or 18 or 20 cm.

In another embodiment, the variable length lines have a length that is a function of k/sin(steering angle), wherein k is a constant.

In another embodiment, which is exemplary and not to be further limiting, scanning outside of the central angular region includes scanning with lines that vary in length between about 1 cm and about 16 cm in length, for example, at least about, 2, or 4, or 6, or 8, or 10, or 12, or 14, or 16, or 18, or 20, or 22, or 24, or 26, or 28, or 30 cm in length.

In another embodiment, scanning outside of the central angular region includes scanning with lines that vary in length between a length of the fixed length lines and about one-third of the length of the fixed length lines.

Another embodiment of the invention provides a medical imaging device for scanning a volume of interest in a subject, the device being designed to scan a central angular region of a volume of interest using a plurality of fixed length lines, and further designed to scan outside of the central angular region of the volume of interest using a plurality of variable length lines, thereby acquiring an image at an improved acquisition rate compared to that of sector scanning using lines of constant length absent variable length lines.

In a further embodiment, the medical imaging device includes a mechanically steered single element transducer. In a further embodiment, the medical imaging device further includes a transducer having a vector array of elements. In another embodiment, the transducer is configured such that an effective apex for scanning is located behind a face of the transducer.

In another embodiment, the medical imaging device is an ultrasound imaging device. In a further embodiment, the medical imaging device is a two-dimensional ultrasound scanner. In a further embodiment, the medical imaging device is a two-dimensional cardiac ultrasound scanner.

In another embodiment, the medical imaging device is a three-dimensional ultrasound scanner. In a further embodiment, the medical imaging device is a three-dimensional cardiac ultrasound scanner.

FIG. 1 is a drawing showing scanning of a heart using traditional fixed line length scanning Arrows represent a subset of the plurality of possible ultrasound beams emitted by the transducer. Because the apex of the heart lies close to the skin, and therefore close to the transducer, a wide angle sector is used to image the entire heart. Further, because the depth of the heart, i.e. distance downward with respect to the transducer, is not uniform, parts of the body other than the heart are also imaged. These unwanted regions are represented schematically by crosshatched triangles.

FIG. 2 is a drawing of an implementation of sector scanning using an array of ultrasound elements. The array of elements is represented by a row of rectangles, and excited elements are the shaded rectangles. One or more elements is excited to form a beam.

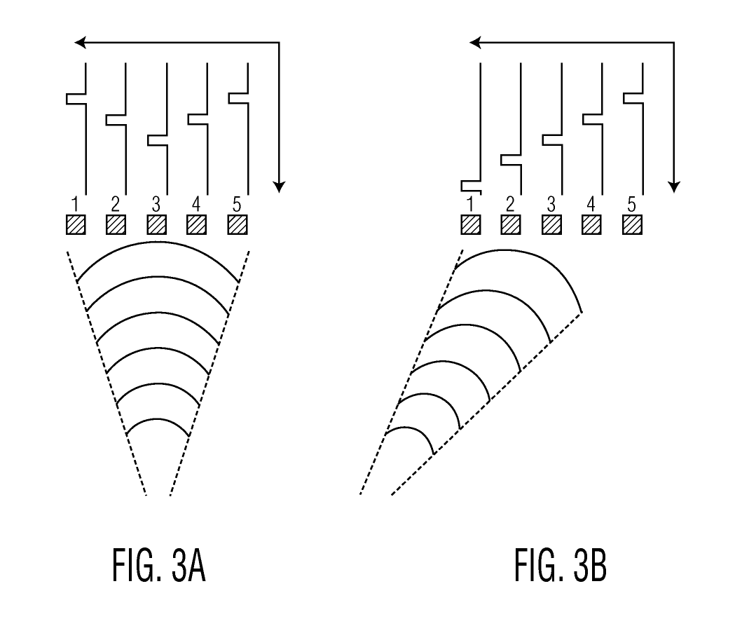

FIG. 3 is a set of drawings that show the formation and steering of beams through use of an array of elements. FIG. 3, panel A shows the focusing of a beam by controlling the timing of the exciting of elements as is well known in the practice of phased array ultrasound. FIG. 3, panel B shows steering of a beam to the one side, e.g. the left as shown, by exciting first element 5, then element 4, then element 3, then element 2, and then element 1.

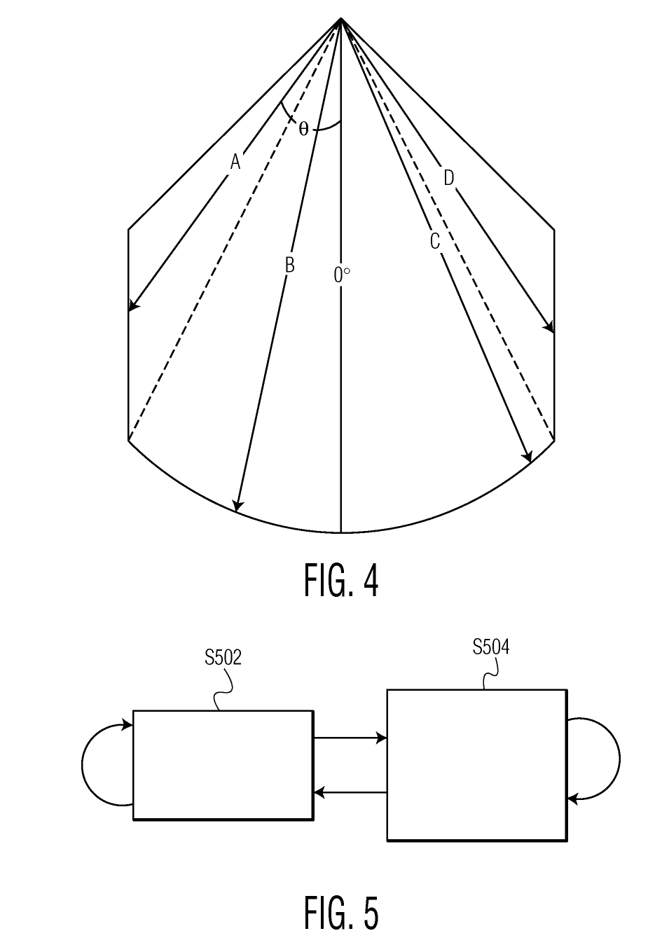

FIG. 4 is a drawing of an embodiment of a variable line length scanning pattern. Dashed lines represent the boundaries of a central angular region. Lines A, B, C and D represent ultrasound beams. Beams within the central angular region (lines B and C) have the same length. Beams outside of the central angular region (lines A and D) have lengths that become shorter as the steering angle (A) increases.

FIG. 5 is a flow chart of an embodiment of the method herein. Step 502 involves scanning a central angular region of a volume of interest with a fixed length line. Step 504 involves scanning outside the central angular region with a variable length line.

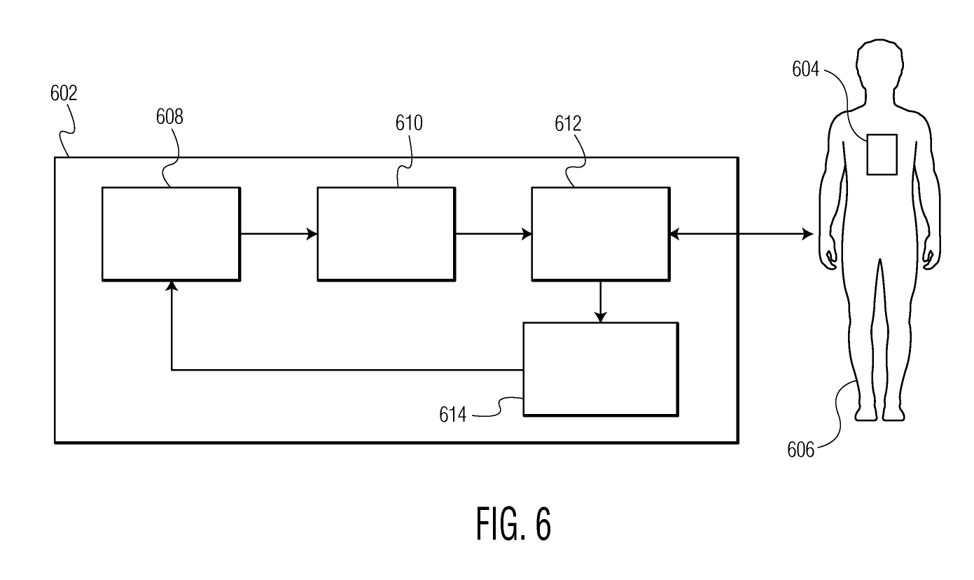

FIG. 6 is a schematic block diagram of an embodiment of an apparatus of the invention herein. A medical imaging device 602 for scanning a volume of interest 604 in a subject 606 includes components which are a control device 608, a pulse generator 610, a transducer 612 and a pre-processor 614.

It is an object of this invention to improve the image acquisition rate during two-dimensional and three-dimensional medical scanning. The methods and apparatus herein are applied to ultrasound scanning applications, such as cardiac scanning, however, other medical imaging devices and other target organs are within the scope of the invention.

Ultrasound imaging systems include a transducer that acts as both a transmitter and a receiver. The transducer consists of either a single element or an array of multiple elements. Ultrasound elements may be made of several materials including, but not limited to: piezocrystals, lead zirconate titanate (PZT), piezo-electric material, and piezo-composite material. In transmission mode, the transducer converts electrical signals into mechanical vibrations, which are transmitted into the body as ultrasound waves. In reception mode, the echoes (backscatter) of the ultrasound waves are converted into electrical signals, then processed.

The two major image formats for ultrasound scanning are linear scanning and sector scanning. Andrew R. Webb, Introduction to Biomedical Imaging 103 (2003), incorporated herein by reference. Sector scanning with an array of ultrasound elements is shown in FIG. 2. Several ultrasound elements, represented in gray, are excited to form a beam. Sector scanning is also possible by mechanically steering a single element. This beam travels through the body in the specified direction and the echoes are received by the transducer. The process is repeated to create an image of a volume of interest.

Beams are formed and steered through the use of constructive interference and the controlling of the phasing of various elements. FIG. 3, panel A shows the focusing of a beam by controlling the timing of the exciting of elements as is well known in the practice of phased array ultrasound. FIG. 3, panel B shows the steering of a beam by exciting element 5, then element 4, then element 3, then element 2, and finally element 1, a method which is also well known.

The term “line” is used herein to represent beams.

Due to the size and shape of target organs, such as the heart, many target volumes of interest do not completely fill the width of a given sector. As a consequence, scanning of the heart or other organ with a conventional ultrasound imaging system produces an image that also includes portions of the body that are of little or no diagnostic value. This inefficiency is exacerbated as the angular width of the sector format is widened to attempt to fully visualize the apex of the heart.

An embodiment of the invention uses sector scanning with variable length lines (VLL). This improves the acquisition rate compared to traditional sector scanning. According to an embodiment, fixed length lines are used in the central angular region. Outside of the central angular region, the line lengths are a function of a steering angle with lines becoming shorter as the steering angle is increased.

As used herein, sector scanning refers to scanning in a polar format with lines of constant length. VLL scanning refers to scanning in a polar format with lines of variable length. FIG. 4 shows an example of VLL scanning.

An example of an implementation of the invention herein is shown in the following table:

| Angles | Line Length |
|---|---|
| −45° to −15° | Line length = constant/sin(steering angle) |
| −15° to +15° | Lines are constant length |
| +15° to +45° | Line length = constant/sin(steering angle) |

As shown in FIG. 4, the effect of VLL scanning is a resulting wide near field and a narrow far field.

FIG. 5 shows a flow diagram for various embodiments of the invention herein. Step 502 involves scanning a central angular region of a volume of interest with a fixed length line. Step 504 involves scanning outside the central angular region with a variable length line. Embodiments of the invention may scan with many different combinations of variable and fixed length lines as described herein.

An advantage of VLL scanning over sector scanning is seen in the following comparison of image acquisition times.

An image acquisition time for a 16 cm 90 degree sector scan with ¾ degree line spacing was achieved in 120*(16*13+40)=29.8 ms (milliseconds), assuming 13 μs/cm (round trip time) and 40 μs of line overhead.

A VLL scan with 16 cm lines in the constant region, a 90 degree near field and ¾ degree line spacing that produces an 8 cm wide “base” required 40*(16*13+40)+80*(9*13+40)=22.5 ms where the scanning time for the variable length lines averaged to the a scanning time of a 9 cm line. In a related embodiment, some additional time is saved in the overhead time for the short lines due to the closer transmit focus, less delay coefficients needed and possibly smaller transmit apertures.

Further, for sector scanning, in order to increase the width, the angle of the sector is increased. In comparison, in order to increase the width in VLL scanning, the horizontal width of the image is increased. In both cases a reduction in frame rate occurs as the area being scanned is increased.

To address a potential increase in reverberation artifacts during VLL scanning in the regions utilizing shorter lines, by reducing and possibly eliminating these artifacts, an embodiment of this invention implements scanning in a non-sequential pattern. In a further embodiment, rather than scanning by incrementally increasing the angular position of each scan line the angular position can alternate or “ping-pong” from one side of the image to the other.

In a further embodiment of the methods and devices herein, VLL scanning is combined with vector array scanning (where the effective apex is placed behind the transducer face) for further improvement in near field width.

Referring now to FIG. 6, additional embodiments of the invention herein include a medical imaging device 602 for scanning a volume of interest 604 in a subject 606. The medical imaging device 602 is used to scan an object and acquire an image.

The medical imaging device includes components which are a control device 608, a pulse generator 610, a transducer 612 and a pre-processor 614. The control device 608 sends signals to the pulse generator 612 to control the power, direction and focus of the output of the medical imaging device 602. The control device adjusts the beams so that a central angular region of the volume of interest 604 is scanned with fixed length lines while regions outside the central angular region are scanned using a plurality of variable length lines when appropriate. The pulse generator 610 receives signals from the control device 608 which define the value of current or voltage for electric pulses. These electric pulses are transmitted to transducer 612 which convert the electric pulses into another form of energy (e.g. ultrasound beams).

This energy is transmitted into a subject 606 where it is reflected and back-scattered as the energy travels through the subject 606 as described herein. The back-scatter of the energy is received by the transducer 612. The energy received by the transducer 612 is sent to the pre-processor 614 for processing such as amplification. Further processing may be conducted in the control device 608.

Additional embodiments include the implementation of the above embodiment in a two-dimensional cardiac ultrasound scanner or a three-dimensional cardiac ultrasound scanner.

It will furthermore be apparent that other and further forms of the invention, and embodiments other than the specific and exemplary embodiments described above, may be devised without departing from the spirit and scope of the appended claims and their equivalents, and therefore it is intended that the scope of this invention encompasses these equivalents and that the description and claims are intended to be exemplary and should not be construed as further limiting.

What is claimed is:

1. A method of imaging of a medical subject, the method comprising:
scanning a central angular region of a volume of interest using a plurality of fixed length lines; and,
scanning outside of the central angular region of the volume of interest using a plurality of variable length lines, thereby acquiring an image at an improved acquisition rate compared to that of sector scanning methods using lines of constant length absent variable length lines.

2. The method according to claim 1, wherein scanning the central angular region and/or scanning outside the central angular region further comprises a non-sequential pattern.

3. The method according to claim 2, wherein the non-sequential pattern comprises alternating an angular position of a beam or a pulse from a first side of the volume of interest to a second side of the volume of interest.

4. The method according to claim 1, wherein scanning the central angular region comprises scanning with constant length lines between the angles of about −15 degrees to about +15 degrees.

5. The method according to claim 1, wherein scanning outside of the central angular region comprises scanning with variable length lines between angles of about −45 degrees to about −15 degrees and about +15 degrees to about +45 degrees.

6. The method according to claim 1, wherein the fixed length lines have a length between about 3 cm and about 30 cm.

7. The method according to claim 1, wherein the fixed length lines have a length of about 16 cm.

8. The method according to claim 1, wherein the variable length lines have a length that is a function of k/sin(steering angle), wherein k is a constant.

9. The method according to claim 1, wherein scanning outside of the central angular region comprises scanning with lines that vary in length between about 1 cm and about 30 cm in length.

10. The method according to claim 1, wherein scanning outside of the central angular region comprises scanning with lines that vary in length between a length of the fixed length lines and about one-third of the length of the fixed length lines.

11. A medical imaging device for scanning a volume of interest in a subject, the device designed to scan a central angular region of a volume of interest using a plurality of fixed length lines; and further designed to scan outside of the central angular region of the volume of interest using a plurality of variable length lines, thereby acquiring an image at an improved acquisition rate compared to that of sector scanning using lines of constant length absent variable length lines.

12. The medical imaging device according to claim 11, wherein the device further comprises a mechanically steered single element transducer.

13. The medical imaging device according to claim 11, wherein the device further comprises a transducer having a vector array of elements.

14. The medical imaging device according to claim 12, wherein the transducer is configured such that an effective apex for scanning is located behind a face of the transducer.

15. The medical imaging device according to claim 11, wherein the device is an ultrasound imaging device.

16. The medical imaging device according to claim 11, wherein the device is a two-dimensional ultrasound scanner.

17. The medical imaging device according to claim 16, wherein the device is a two-dimensional cardiac ultrasound scanner.

18. The medical imaging device according to claim 11, wherein the device is a three-dimensional ultrasound scanner.

19. The medical imaging device according to claim 18, wherein the device is a three-dimensional cardiac ultrasound scanner.
